(19) United States
(12) Patent Application Publication
(10) Pub. No.: US 2021/0221794 A1
(43) Pub. Date: Jul. 22, 2021

(54) ISATIN DERIVATIVES

(71) Applicant: KING SAUD UNIVERSITY, RIYADH (SA)

(72) Inventors: AHMED BARI, RIYADH (SA); SAEED ALI SYED, RIYADH (SA); MOHAMMAD ABDULAZIZ ALTAMIMI, RIYADH (SA)

(21) Appl. No.: 16/747,297

(22) Filed: Jan. 20, 2020

(51) Int. Cl. C07D 403/12; A61P 35/00

(52) U.S. Cl. CPC C07D 403/12; A61P 35/00

(57) ABSTRACT

Isatin derivatives having a chemical structure according to Formula I:

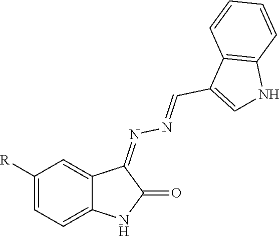

where R represents a hydrogen or chloro group, or a pharmaceutically acceptable salt thereof have anticancer activities. The isatin derivatives have anticancer activities and may be synthesized and used in a pharmaceutical composition or otherwise to treat cancers in a subject diagnosed with, for example, at least one of leukemia, lung cancer, central nervous system (CNS) cancer, melanoma, ovarian cancer, renal cancer, prostate cancer, breast cancer or colon cancer.

### BACKGROUND

### 1. Field

The disclosure of the present patent application relates to compounds useful as anticancer agents, and particularly to anticancer agents based on 1H-indole-2, 3-dione derivatives and methods for their preparation and use.

### 2. Description of the Related Art

Cancer is one of the leading causes of death in the world, primarily characterized by a loss of control of cell growth in any cell type, ultimately leading to death. Chemotherapy remains one of the primary modalities for the treatment of cancer. However, the use of available chemotherapeutics is limited mainly due to drug resistance and toxicity. Resistance to chemotherapies and damage due to chemotherapy toxicity limit the use of existing chemotherapeutic drugs. Combinations of chemotherapies are often pursued, as targeting different proteins increases chemotherapeutic efficiency, antagonizes resistance development and decreases toxicity effects.

1H-indole-2,3-dione or isatin is a heterocyclic natural product found in plants of the genus Isatis, and is also present in humans as a metabolic derivative of adrenaline. Isatin is a versatile synthetic intermediate with various pharmacological properties.

Thus, isatin derivatives useful as anticancer agents solving the aforementioned problems are desired.

### SUMMARY

Anticancer agents of the present disclosure are isatin derivatives comprising an indole moiety attached to isatin via a carbon to nitrogen bond formation. The isatin derivatives have a chemical structure according to Formula I:

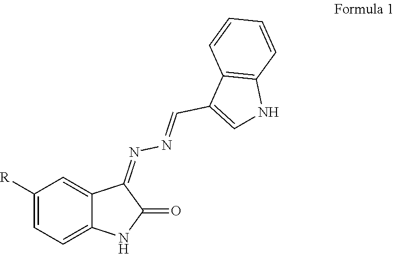

where R represents a hydrogen or chloro group, or a pharmaceutically acceptable salt thereof.

Other embodiments include pharmaceutical compositions comprising the above compounds, methods of synthesizing such compounds and pharmaceutical compositions and methods of treatment using the compounds and pharmaceutical compositions.

These and other features of the present subject matter will become readily apparent upon further review of the following specification.

### BRIEF DESCRIPTION OF THE DRAWINGS

FIG. 1 depicts a synthesis scheme of 3-((1H-indol-3-yl)methylene)hydrazono)indolin-2-one [referred to herein by National Institute of Health (NIH) compound code T1] and 5-chloro-3-((1H-indol-3-yl)methylene)hydrazono)indolin-2-one—(referred to herein by NIH compound code T2).

FIG. 2A provides levels of anticancer activities of the isatin derivative T1 for a diverse range of model cancer cell strains.

FIG. 2B provides levels of anticancer activities of the isatin derivative T2 for a diverse range of model cancer cell strains.

Similar reference characters denote corresponding features consistently throughout the attached drawings.

### DETAILED DESCRIPTION OF THE PREFERRED EMBODIMENTS

The isatin derivatives of the present disclosure have a chemical structure according to Formula I:

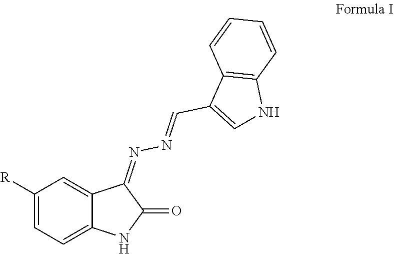

where R represents a hydrogen or chloro group, or a pharmaceutically acceptable salt thereof.

Other embodiments of the present subject matter include a pharmaceutical composition comprising one or more of the isatin derivatives and a pharmaceutically acceptable carrier.

The isatin derivatives can be used for treating a patient suffering from cancer. A method of treating a patient suffering from cancer can include the step of administering a pharmaceutical composition including an effective amount of at least one isatin derivative to a patient in need thereof. The cancer can include at least one of leukemia, lung cancer, central nervous system (CNS) cancer, melanoma, ovarian cancer, renal cancer, prostate cancer, breast cancer and colon cancer. In an embodiment, prior to administering the pharmaceutical composition, the method can include preparing the isatin derivative and determining an appropriate dosing regimen for administering the isatin derivative. The pharmaceutical composition can be administered pursuant to the dosing regimen.

As described in the following Examples, the isatin derivatives can inhibit growth of cancer cells. Accordingly, the methods of treating a cancer patient according to embodiments discussed above may be combined with any existing cancer therapy, including surgery, radiation or additional chemotherapy or immune therapy.

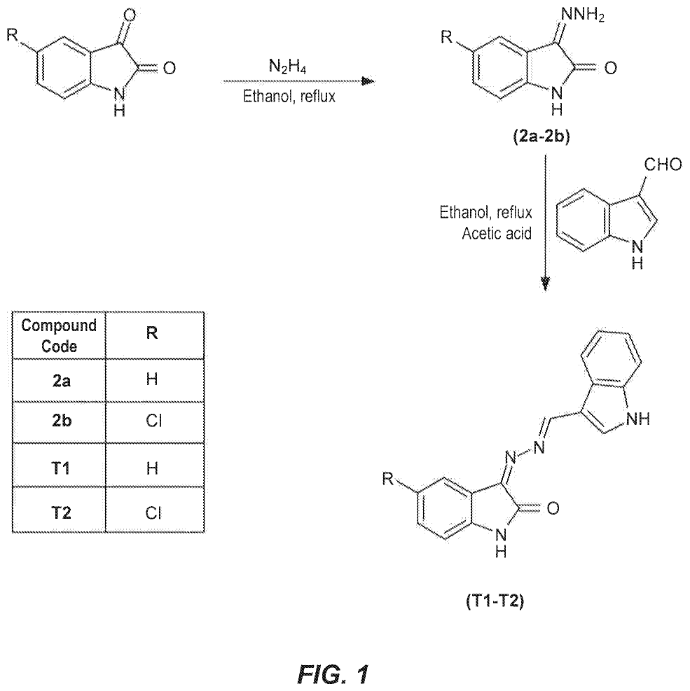

The isatin derivatives may be synthesized as illustrated in the scheme shown in FIG. 1. Referring to FIG. 1, 3-((1H-indol-3-yl)methylene)hydrazono)indolin-2-one (T1) can be prepared by the reaction of 3-hydrazonoindolin-2-one in ethanol with indole-3-carboxaldehyde. 5-chloro-3-((1H-indol-3-yl)methylene)hydrazono)indolin-2-one (T2) may be prepared by the reaction of 5-chloro-3-hydrazonoindolin-2-one in ethanol with indole-3-carboxaldehyde. A catalytic amount of acetic acid can be added at room temperature to complete the reaction. The resulting mixtures can be refluxed with stirring. Upon evaporation of the solvent, the resulting solid may be washed and recrystallized to produce the isatin derivatives.

Exemplary samples of the isatin derivatives were synthesized, as described in the following examples, and the structures of all of the synthesized compounds were determined by nuclear magnetic resonance (NMR), Fourier transform infrared spectroscopy (FTIR), mass spectroscopy (MS), and elemental analysis.

It should be understood that the amounts of materials for the methods described herein are exemplary, and appropriate scaling of the amounts are encompassed by the present subject matter, as long as the relative ratios of materials are maintained. As used herein, the term “about,” when used to modify a numerical value, means within ten percent of that numerical value.

The pharmaceutical compositions may include different concentrations of the isatin derivatives. Diluting solvents may include, for example, water, saline or alcohol.

A pharmaceutically acceptable salt includes hydrochlorides, carbonates, bicarbonates, benzene sulfonate, benzoate, gluconate, mesylate, acetate, phosphate and p-toluene sulfate salts, which are generally prepared by the reaction of free acid with a suitable base. The base may be either organic or inorganic.

The isatin derivatives can be administered by any acceptable route, including oral, intravenous, transdermal, directly to the region requiring chemotherapy by any chemotherapeutic means. As a further alternative, the isatin derivatives may be administered as liposome formulations. Liposomes are phospholipids based vesicles which may enclose the isatin derivatives. Liposomes loaded with the isatin derivatives can be dispersed in aqueous medium, which may contain stabilizers, preservatives and excipients.

It should be understood that the pharmaceutical compositions can include one or more of the isatin derivatives or pharmaceutically equivalent salts thereof. One or more of the isatin derivatives can be mixed with a pharmaceutically acceptable excipient as per acceptable pharmaceutical compounding procedures. Excipients include, but are not limited to, binders, suspending agents, lubricants, flavoring, sweeteners, preservatives, dyes and coatings. In preparation of liquid oral dosage forms, any pharmaceutical carriers may be used, such as water, glycerol, alcohols, preservatives and coloring agents. In solid dosage forms, carriers include, but are not limited to, starches, sugars, granulating agents and binders. Injectable preparations may also be prepared, for which acceptable pharmaceutical carriers include, but are not necessarily limited to alcohol, dimethylsulfoxide (DMSO), saline. Other potential formulations may include nanoparticle formulations, micellar formulations, biodegradable formulations and water soluble formulations.

Pharmaceutical compositions for intravenous injections can include sterile water, preservatives, wetting agents, excipients and dispersion agents. Various antibacterial and antifungal agents may be used to prevent microbial growth, including paraben, chlorobutanol, and ethanol. Gelatin may be used for prolonged absorption of the isatin derivatives in the body. For slow release of the isatin derivatives, crystalline or materials with poor water solubility may be used.

The present compositions may be formulated as tablets, pills, capsules, powders, ampules, sterile solutions and auto injector modules, for example, and in various concentrations. The isatin derivatives may be mixed with a pharmaceutically acceptable carrier or excipient appropriate for the formulation. A therapeutically effective dose or an amount of the isatin derivative or pharmaceutical composition may be determined initially according to the nature of the cancer and the organ affected.

The following examples illustrate the present teachings.

### EXAMPLES

### Example 1

### Synthesis of the Isatin Derivatives

Exemplary samples of isatin derivatives T1 and T2 were prepared by the reaction of 2a (1 mmol) and 2b (1 mmol), respectively, in ethanol (50 mL) with indole-3-carboxaldehyde (1.1 mmol). Addition of a catalytic amount of acetic acid at room temperature facilitated completion of the reaction. The resulting mixtures were refluxed for 3 hours with stirring. The solvents of the refluxed mixtures were evaporated in vacuo to produce a solid. The solid was washed with cold water several times and recrystallized with ethanol to afford the exemplary samples, characterized as below.

3-((1H-indol-3-yl)methylene)hydrazono)indolin-2-one (labeled as T1): Color: Orange. Yield: 82%, M.P. 249-251° C., FTIR (KBr): ν=cm⁻¹, 1660 (C═N), 1717 (C═O), 3259 (NH). ¹HNMR (700.174 MHz, DMSO-d): δ=6.92 (d, 1H, Ph), 6.93 (d, 1H, Ph), 7.10 (m, 3H, Ph), 7.58 (d, 1H, Ph), 8.25 (t, 1H, Ph), 8.42 (s, 1H, CH), 8.97 (s, 1H, CH), 10.79 (s, 1H, NH), 12.19 (s, 1H, NH). ¹³C NMR (125.76 MHz, DMSO-d₆): δ=111, 112.6, 113.1, 117.6, 121.7, 121.4, 122.5, 123.9, 124.9, 128.1, 133.2, 136.9, 138, 144.7, 149.5, 162.4, 165.8. MS (EI): m/z (%)=289 (39) (M+). —C₁₇H₁₂N₄O (288.10): calcd. C, 70.82, H, 4.20, N, 19.43; found C, 70.71, H, 4.30, N, 19.67.

5-chloro-3-((1H-indol-3-yl)methylene)hydrazono)indolin-2-one (labeled as T-2): Color: Orange. Yield: 78%, M.P. 279-281° C., FTIR (KBr): ν=cm⁻¹, 1671 (C═N), 1721 (C═O), 3230 (NH). ¹HNMR (700.174 MHz, DMSO-d₆): δ=6.87 (d, 1H, Ph), 6.94 (m, 2H, Ph), 7.34 (m, 1H, Ph), 7.60 (d, 1H, Ph), 8.30 (dd, 2H, Ph), 8.54 (t. 1H, CH), 9.03 (s, 1H, CH), 10.91 (s, 1H, NH), 12.28 (s, 1H, NH, ¹³C NMR (125.76 MHz, DMSO-d₆): δ=110, 112.3, 112.8, 117.6, 121.2, 121.7, 122.5, 124.1, 124.9, 128.1, 133.5, 136.9, 138, 144.7, 149.5, 162.4, 165.8. MS (EI): m/z (%)=323 (72) (M+). —C₁₇H₁₁ClN₄O (322.06): calcd. C, 63.26, H, 3.44, N, 17.36, found C, 63.10, H, 3.31, N, 17.42.

### Example 2

### Methodology of In Vitro Anticancer Screening

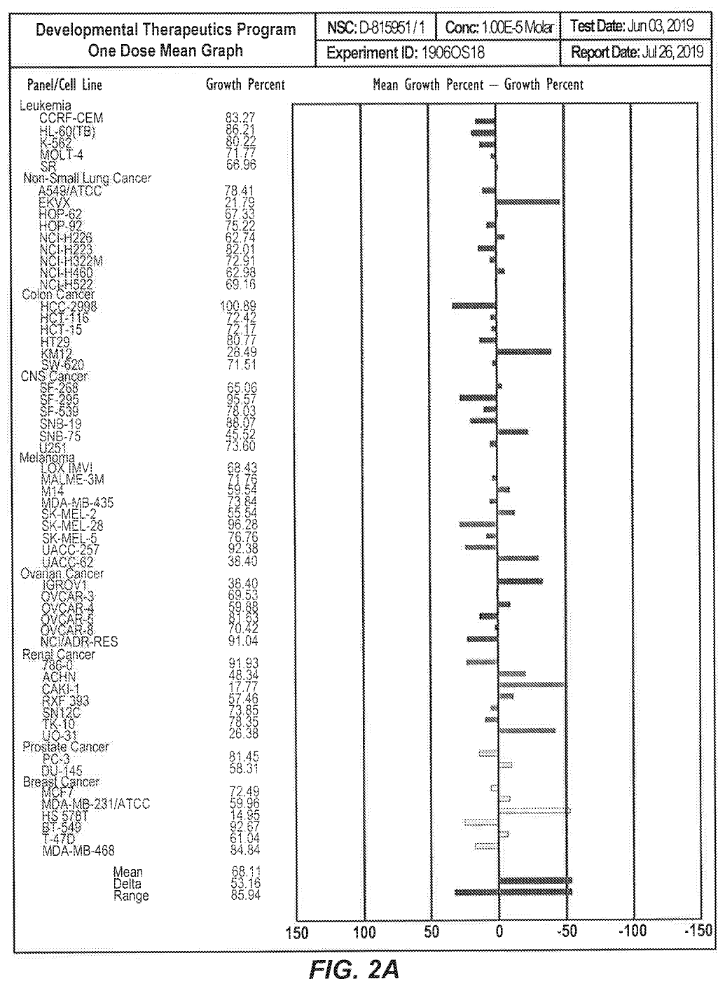

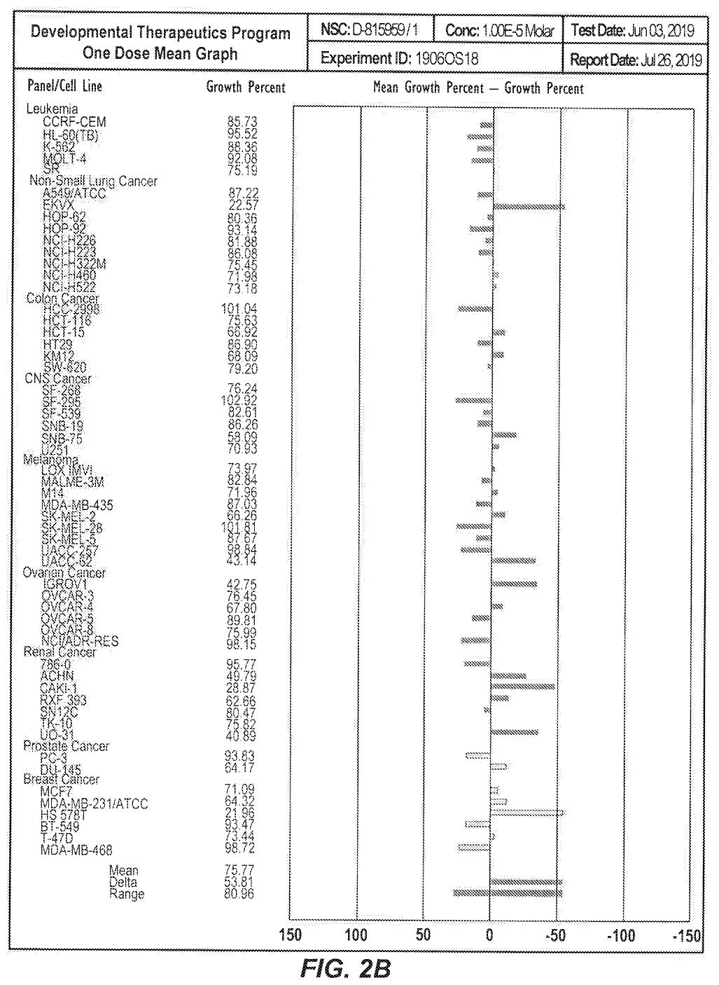

Anticancer activities of the exemplary isatin derivatives were tested by the NIH National Cancer Institutes (NCI), with results summarized in FIGS. 2A-2B. The anticancer activity was tested by NCI, division of cancer treatment and diagnosis, according to the NCI-60 Human Tumor Cell Lines Screen, one-dose screen (https://dtp.cancer.gov/discovery_development/nci-60/methodology.htm). This screening allows for qualified synthesized chemicals to be subjected to an anticancer test against 60 different cancer cell lines. Briefly, exemplary T1 or T2 was dissolved in DMSO:Glycerol, 9:1, at 4 mmol and kept frozen prior to use. For inoculation, a 96 well microtiter was used for the study of different cell lines. Based on the doubling time for each cell line, the well density varied. The plates were incubated at 37° C., 5% CO₂, 95% air and 100% relative humidity for 1 day. The exemplary T1 or T2 or a control were added to the plates at a concentration of 10-M. After 2 more days, the plates were fixed and stained to identify growth inhibition relative to cells without drug treatment. Anti-proliferative activity was determined based on careful analysis of historical DTP screening data. Cell death and net growth inhibition were determined based on a time zero control. A value of 100 indicates no growth inhibition. A value of 40 indicates 60% growth inhibition. A value of 0 indicates no net growth over the course of the experiment. A value of −40 indicates 40% lethality. A value of −100 indicates that all cells are dead.

As shown in FIG. 2A, T1 exhibited a range of anticancer activities against the 60 different cancer cell lines tested. The most significant anticancer activities compared to control were achieved for Non-Small Cell Lung Cancer cells (EKVX) (˜79% reduction), Colon Cancer cells (KM12) (˜72% reduction), Ovarian Cancer cells (IGROV1) (˜65% reduction), Renal Cancer cells (CAKI-1, UO-31) (˜83%, ˜74% reduction, respectively) and Breast Cancer cells (HS 578T) (˜85% reduction).

As shown in FIG. 2B, T2 exhibited a range of anticancer activities against the 60 different cancer cell lines tested. The most significant anticancer activities compared to control were achieved for Non-Small Cell Lung Cancer cells (EKVX) (˜77% reduction), Renal Cancer cells (CAKI-1, UO-31) (˜71%, ˜59% reduction, respectively) and Breast Cancer cells (HS 578T) (˜78% reduction).

T1 showed particularly significant activity against Non-Small Cell Lung Cancer (EKVX), Colon Cancer (KM12), Ovarian Cancer (IGROV1), Renal Cancer (CAKI-1, UO-31) and Breast Cancer (HS 578T) respectively. T2 showed particularly significant activity against Non-Small Cell Lung Cancer (EKVX), Renal Cancer (CAKI-1, UO-31) and Breast Cancer (HS 578T). Each of T1 and T2 showed additional activities against other cell types, as depicted in FIGS. 2A-2B.

It is to be understood that the isatin derivatives and related compositions and methods are not limited to the specific embodiments described above, but encompasses any and all embodiments within the scope of the generic language of the following claims enabled by the embodiments described herein, or otherwise shown in the drawings or described above in terms sufficient to enable one of ordinary skill in the art to make and use the claimed subject matter.

1. An isatin derivative having the following structural formula: 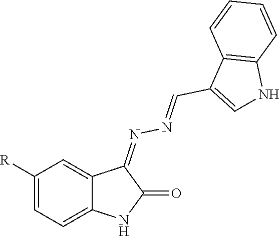
where R is selected from the group consisting of hydrogen and a chloro group,
or a pharmaceutically acceptable salt thereof.

2. The isatin derivative of claim 1, wherein R is hydrogen.

3. The isatin derivative of claim 1, wherein R is a chloro group.

4. A method of treating cancer, comprising the step of administering an effective amount of the isatin derivative of claim 1 to a patient in need thereof.

5. The method of claim 4, wherein the cancer comprises at least one of leukemia, lung cancer, central nervous system (CNS) cancer, melanoma, ovarian cancer, renal cancer, prostate cancer, breast cancer, and colon cancer.

6. The method of claim 4, wherein R is hydrogen and the cancer comprises at least one of non-small cell lung cancer, colon cancer, ovarian cancer, renal cancer, and breast cancer.

7. The method of claim 4, wherein R is a chloro group and the cancer comprises at least one of non-small cell lung cancer, renal cancer, and breast cancer.

8. A pharmaceutical composition comprising an isatin derivative and a pharmaceutically acceptable excipient, wherein the isatin derivative is a compound having a formula: 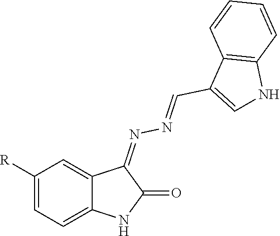
where R is selected from the group consisting of hydrogen and a chloro group,
or a pharmaceutically acceptable salt thereof.

9. The pharmaceutical composition of claim 8, wherein R is hydrogen.

10. The pharmaceutical composition of claim 8, wherein R is a chloro group.

11. A method of treating cancer, comprising the step of administering an effective amount of the pharmaceutical composition of claim 8 to a patient in need thereof.

12. The method of claim 11, wherein the cancer comprises at least one of leukemia, lung cancer, central nervous system (CNS) cancer, melanoma, ovarian cancer, renal cancer, prostate cancer, breast cancer, and colon cancer.

13. The method of claim 11, wherein R is hydrogen and the cancer comprises at least one of non-small cell lung cancer, colon cancer, ovarian cancer, renal cancer and breast cancer.

14. The method of claim 11, wherein R is a chloro group and the cancer comprises at least one of non-small cell lung cancer, renal cancer, and breast cancer.

15. A method of synthesizing an isatin derivative for treating a patient suffering from cancer, the isatin derivative having the following structural formula: 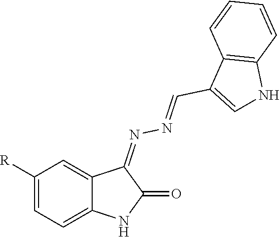
wherein R is hydrogen or a chloro group, or a pharmaceutically acceptable salt thereof, wherein the method comprises steps of:
dissolving a precursor of an isatin derivative in ethanol to form a solution, wherein the precursor is selected from the group consisting of 3-hydrazonoindolin-2-one and 5-chloro-3-hydrazonoindolin-2-one;
mixing the solution with indole-3-carboxaldehyde;
adding a catalytic amount of acetic acid to form a reaction mixture;
refluxing the reaction mixture for three hours with stirring;
evaporating a solvent in the mixture to produce a solid comprising the isatin derivative; and
washing the solid with water and recrystallizing the washed solid with ethanol to isolate the isatin derivative,
whereby the resultant compound can be used to treat patients with cell lines non-small cell lung cancer (EKVX), colon cancer (KM12), ovarian cancer (IGROV1), renal cancer (CAKI-1, UO-31) and breast cancer (HS 578T).

16-18. (canceled)
